(19) United States
(12) Patent Application Publication
(10) Pub. No.: US 2007/0021505 A1
(43) Pub. Date: Jan. 25, 2007

(54) Prevention and treatment of ophthalmic complications of diabetes

(75) Inventors: Rajiv Bhushan, Palo Alto, CA (US); Jerry B. Gin, Sunnyvale, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(73) Assignee: Chakshu Research, Inc, Los Gatos, CA (US)

(21) Appl. No.: 11/486,502

(22) Filed: Jul. 14, 2006

Related U.S. Application Data
(60) Provisional application No. 60/699,929, filed on Jul. 15, 2005.

(51) Int. Cl. A61K 31/198; A61K 31/185

(52) U.S. Cl. 514/553; 514/566

(57) ABSTRACT

An method and formulation are provided for the prevention and treatment of adverse ocular conditions which are complications of diabetes. In one embodiment, the invention comprises administering to a person having diabetes, insulin resistance, or a risk factor for diabetes a formulation comprising a metal chelator and a transport enhancer. Most preferably, the metal chelator is EDTA or a salt of EDTA, and the transport enhancer is methylsulfonylmethane (MSM). The formulation may be in a form suitable for application to the eye itself, for example, in the form of eye drops.

### CROSS REFERENCE TO RELATED APPLICATIONS

This application claims priority under 35 U.S.C. §119(e)(1) to U.S. Provisional Application Ser. No. 60/699,929, filed Jul. 15, 2005, the disclosure of which is incorporated herein by reference in its entirety.

### TECHNICAL FIELD

This invention relates generally to the treatment of ocular disorders, ocular diseases, and other adverse ocular conditions. More particularly, the invention pertains to an ophthalmic formulation for the treatment the ophthalmic complications of diabetes.

### BACKGROUND

In the majority of people blood glucose is under fairly tight physiological control. Glucose resulting from digestion of a meal is rapidly taken up and stored in muscle, fat, and liver cells, so that the release of glucose into the blood resulting from digestion of a meal does not result in an undue elevation of the concentration of glucose in the blood. The hormone insulin is the chemical messenger which causes the muscle, fat, and liver cells to take up glucose.

In certain individuals the physiological control of blood glucose breaks down. In some persons it breaks down because the beta cells of the pancreas, which produce insulin, become unable to produce it in normal quantities. In other persons, the responsiveness of the cells to insulin becomes progressively less, and so eventually, even though the pancreas produces large quantities of insulin to compensate for the body's diminished responsiveness, the takeup of glucose becomes insufficient to keep blood levels of glucose regulated. The first of these conditions is referred to as type 1 diabetes; the second, as type 2 diabetes. For general information on the physiology of diabetes, one may for example consult chapter 78 of Arthur C. Guyton & John E. Hall, Textbook of Medical Physiology (10th ed. 2000).

An excess of glucose has a number of adverse consequences for cells of the human body. Diabetic complications manifest themselves in blood vessels, in nervous system cells, and in other areas. In the eye, two complications are notable. Diabetic retinopathy results in degeneration of the vasculature of the retina which can damage or destroy the retina. It is a major cause of blindness, said to be the reason for 25% of the registrations for blindness in the western world. Cataract is substantially more likely to occur in diabetics than in non-diabetics. It has been described as one of the earliest secondary complications of diabetes. Other ophthalmic effects of diabetes include neuropathies affecting the innervation of the eye. It has also been widely suggested that diabetes is a risk factor for glaucoma.

Cataract is an opacification of the lens of the eye. The lens is a unique structure within the body, for example because its proteins are very long lived and rarely renewed. In the present state of therapy, the treatment of cataracts depends upon the correction of vision using eyeglasses, contact lenses, or surgical operations such as insertion of an intra-ocular lens into the capsula lentis after extra-capsular cataract extraction. There has been a great deal of interest in the development of a pharmacological treatment, both because of the cost of surgery and because of the less desirable characteristics of artificial lenses compared to the human lens. There is also a great deal of interest in the development of drugs which might prevent or delay the formation of diabetic cataract. For further information please refer to Z. Kyselova et al., “Pharmacological prevention of diabetic cataract,” Journal of Diabetes and its Complications, 18, 129-140 (2004).

The lens, which is what cataract affects, is primarily protein. It contains many cells which have lost their nuclei and other internal organs. In general the outer cells of the lens proliferate and migrate inwards, pushing other cells towards the center of the lens. Unlike many other proteins the body which turn over rapidly, the proteins in the lens last for long periods of time on the order of decades. For this reason there is a significant possibility of harm to the lens from conditions which can cause degeneration of these proteins, without the same possibilities of recovery from the harm that might exist in other tissues with a higher protein turnover. Lenses with cataract are characterized by protein aggregates that scatter light and reduce transparency.

Diabetic retinopathy manifests itself primarily in the blood vessels. In earlier stages of the retinopathy it is common to see microaneurysms and blockages of the vasculature. In later stages, there is a proliferation of the blood vessels of the retina. It is believed that this proliferation results from the eye's reaction to a lack of blood flow caused by the blockages occurring earlier. The effect of excessively high glucose levels on the small blood vessels of the retina may be related to its effect on blood vessels elsewhere in the body. Diabetes is a factor which is also known to increase the likelihood of atherosclerosis in larger blood vessels.

The association of diabetic complications, in the eye and elsewhere, with oxidative stress has been widely studied. The normal operation of the body's metabolism produces a variety of reactive oxygen species (ROS's). Reactive oxygen species include for example hydroxyl ion OH⁻and hydrogen peroxide H₂O₂. The body contains a number of mechanisms to remove these species, limiting their action within the body so that they do not damage the body's constituents. Oxidative stress occurs when an excessive amount of ROS's is produced, or when the mechanisms for removing ROS's are overloaded and are unable to remove them as required for normal functioning of the body.

It is generally believed that oxidative stress is a mechanism by which the diabetic complications occur. There is considerable study being made of the precise biochemical and molecular mechanisms by which the oxidative stress occurs and acts. There have been proposals to treat the complications of diabetes with antioxidants such as vitamins C and E.

As alluded to above, current therapeutic attempts to address many ocular disorders and diseases, including aging-related ocular problems, often involve surgical intervention. Surgical procedures are, of course, invasive, and, furthermore, often do not achieve the desired therapeutic goal. Additionally, surgery can be very expensive and may result in significant undesired after- effects. For example, secondary cataracts may develop after cataract surgery and infections may set in. Endophthalmitis has also been observed after cataract surgery. In addition, advanced surgical techniques are not universally available, because they require a very well developed medical infrastructure. Therefore, it would be of significant advantage to provide straightforward and effective pharmacological therapies that obviate the need for surgery.

It is therefore an objective of the present invention to provide a formulation which allows pharmacologic prevention and/or treatment of the ophthalmic complications of diabetes.

### SUMMARY OF THE INVENTION

In an embodiment of the invention, a method is provided for the prevention and treatment of the ocular complications of diabetes. The method may be applied to persons who have been diagnosed with diabetes, or to persons who manifest insulin resistance, or to persons who have a risk factor for diabetes. The methods comprise administration of a pharmaceutical formulation comprising an effective amount of a biocompatible metal complexer, combined with a transport enhancer, in a pharmaceutically acceptable carrier.

In another embodiment of the invention, a sterile ophthalmic formulation is provided whose active ingredients are a biocompatible metal complexer and a transport enhancer. The formulation may contain an optional additional transport enhancer. The formulation also contains a pharmaceutically acceptable carrier and may contain other optional excipients.

The ophthalmic formulation may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix.

The invention also pertains to ocular inserts for the controlled release of a biocompatible metal complexer as noted above. The insert may be a gradually but completely soluble implant, such as may be made by incorporating swellable, hydrogel-forming polymers into an aqueous liquid formulation. The insert may also be insoluble, in which case the agent is released from an internal reservoir through an outer membrane via diffusion or osmosis.

### BRIEF DESCRIPTION OF THE FIGURES

FIG. 1 depicts lenses from rats studied in Example 1, some of which were treated with a method of the invention.

FIG. 2 depicts lenses from rats studied in Example 2, some of which were subjected to a formulation of the invention.

FIG. 3 depicts the % transmission of light through lenses of Example 2 for the different treatments.

FIG. 4 depicts the effect of MSM and MSM/EDTA on human lens epithelial cells subjected to glucose-induced toxicity, as discussed in Example 3.

### DETAILED DESCRIPTION OF EMBODIMENTS OF THE INVENTION

Unless otherwise indicated, the invention is not limited to specific formulation types, formulation components, dosage regimens, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a metal complexer” includes a single such complexer as well as a combination or mixture of two or more different metal complexers, reference to “a transport enhancer” includes not only a single transport enhancer but also a combination or mixture of two or more different transport enhancers, reference to “a pharmaceutically acceptable ophthalmic carrier” includes two or more such carriers as well as a single carrier, and the like.

In this specification and in the claims that follow, reference will be made to a number of terms, which shall be defined to have the following meanings:

When referring to a formulation component, it is intended that the term used, e.g., “agent,” encompass not only the specified molecular entity but also its pharmaceutically acceptable analogs, including, but not limited to, salts, esters, amides, prodrugs, conjugates, active metabolites, and other such derivatives, analogs, and related compounds.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause. Unless otherwise indicated herein, either explicitly or by implication, if the term “treatment” (or “treating”) is used without reference to possible prevention, it is intended that prevention be encompassed as well, such that “a method for the treatment of diabetic cataract” would be interpreted as encompassing “a method for the prevention of diabetic cataract.”

By the terms “effective amount” and “therapeutically effective amount” of a formulation or formulation component is meant a nontoxic but sufficient amount of the formulation or component to provide the desired effect.

The term “controlled release” refers to an agent-containing formulation or fraction thereof in which release of the agent is not immediate, i.e., with a “controlled release” formulation, administration does not result in immediate release of the agent into an absorption pool. The term is used interchangeably with “nonimmediate release” as defined in Remington: The Science and Practice of Pharmacy, Nineteenth Ed. (Easton, PA: Mack Publishing Company, 1995). In general, the term “controlled release” as used herein refers to “sustained release” rather than to “delayed release” formulations. The term “sustained release” (synonymous with “extended release”) is used in its conventional sense to refer to a formulation that provides for gradual release of an agent over an extended period of time.

By “pharmaceutically acceptable” is meant a component that is not biologically or otherwise undesirable, i.e., the component may be incorporated into an ophthalmic formulation of the invention and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component is one which is suitable for use as an excipient in the preparation of pharmaceutical preparations of the type being considered. For example, an inactive ingredient would generally be considered “pharmaceutically acceptable” if it is included on the Inactive Ingredient Guide prepared by the U.S. Food and Drug Administration.

The phrase “having the formula” or “having the structure” is not intended to be limiting and is used in the same way that the term “comprising” is commonly used.

The term “alkyl” as used herein refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc.

The term “alkoxy” as used herein intends an alkyl group bound through a single, terminal ether linkage; that is, an “alkoxy” group may be represented as —O-alkyl where alkyl is as defined above.

The term “aryl,” as used herein and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. If not otherwise indicated, the term “aryl” includes unsubstituted and substituted aryl, wherein the substituents may be as set forth above with respect to optionally substituted “alkyl” groups.

The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “acyl” refers to substituents having the formula —(CO)-alkyl, —(CO)-aryl, or —(CO)-aralkyl, wherein “alkyl,” “aryl, and “aralkyl” are as defined above.

The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

In an embodiment of the invention, a method is provided for the prevention and treatment of the ocular complications of diabetes. The method may be applied to persons who have been diagnosed with diabetes, or to persons who manifest insulin resistance, or to persons who have a risk factor for diabetes. The methods comprise administration of a pharmaceutical formulation comprising an effective amount of a biocompatible metal complexer, combined with a transport enhancer, in a pharmaceutically acceptable carrier.

There has been considerable study of risk factors for diabetes. Obesity, for example, is strongly associated with type 2 diabetes. It is believed that diabetes has a significant genetic component. Thus, a family history of diabetes is also a risk factor. Particular ethnic groups have been identified as having a higher incidence of diabetes. High blood pressure, abnormal blood lipids, and a lack of exercise are also viewed as risk factors for diabetes. In the United States, a range of blood glucose concentrations has been formally defined as “pre-diabetes.”

In formulations of the invention, the metal complexer and transport enhancer may preferably be administered topically to the eye. In that case, these ingredients may be applied to the eye in any form suitable for ocular drug administration, e.g., as a solution or suspension for administration as eye drops or eye washes, as an ointment, or in an ocular insert that can be implanted in the conjunctiva, sclera, pars plana, anterior segment, or posterior segment of the eye. Implants provide for controlled release of the formulation to the ocular surface, typically sustained release over an extended time period.

Metal complexers can be divided into two general categories: chelators and complexing ligands.

The word chelator comes from the Greek word “chele” which means “claw” or “pincer.” As the name implies, metals that are complexed with chelators form a claw-like structure consisting of one or more molecules. The metal chelate structure is circular, generally containing 5 or 6 member rings that are structurally and chemically stable.

Chelators can be classified by two different methods. One method is by their use: they may be classified as extraction type and color-forming type. Extractions with chelators may be for preparative or analytical purposes. The chelating extraction reaction generally consists of addition of a chelator to a metal-containing solution or material to selectively extract the metal or metals of interest. The color-forming type of chelators—including pyridylazonaphthol (PAN), pyridylazoresorcinol (PAR), thioazoylazoresorcinol (TAR), and many others—have been used in analytical chemistry for many years. The chemistry is similar to that of the extraction type, except that the color-forming chelator will form a distinctive color in the presence or absence of a targeted metal. Generally the types of functional groups that form the chelate complex are similar; however, a color-forming chelator will be water soluble due to the addition of polar or ionic functional groups (such as a sulfonic acid group) to the chelating molecule.

Another method of classifying chelators is according to whether or not the formation of the metal chelate complex results in charge neutralization. Chelators generally contain hydronium ions (from a carboxylic acid or hydroxy functional group) which result in charge neutralization, e.g., 8-hydroxyquinoline. But they may also be non-ionic and simply add to the metal conserving the charge of the metal, e.g., ethylene diamine or 1,10-phenanthroline. Chelators usually have one acid group and one basic group per ring structure. Typical acid groups are carboxylic acid, hydroxyl, (phenolic or enolic), thiol, hydroxylamine, and arsonic acids. Typical basic groups include ketone and primary, secondary, and tertiary amine groups. Virtually all organic functional groups have been incorporated into chelators.

A complexing ligand does not form a ring structure, but still can form strong complexes of the ligand and metal. An example of a complexing ligand is cyanide which can form strong complexes with certain metals such as Fe³⁺ and Cu²⁺. Free cyanide is used to complex and extract gold metal from ore. One or more of the ligands can complex with the metals depending on the ligand and ligand concentration. Silver forms 3 different complexes of 1 silver molecule to 2, 3 or 4 cyanide molecules depending on the cyanide concentration, but gold forms only 1 cyanide complex of 1 gold molecule and 2 cyanide molecules. Other complexing ligands include chloride, bromide, iodide, thiocyanate, and many others.

It is possible to add selectivity to the complexation reaction. Some chelators are very selective for a particular metal. For example, dimethylglyoxime forms a planar structure with Ni²⁺ and selectively extracts the metal. Selectivity can be moderated by adjusting the pH. In the case where an acidic group is present, the chelator is made more general by increasing pH and more selective by decreasing the pH. Only metals that form the strongest chelators will form metal chelates under increasingly acidic conditions.

Chelating or ligand complexers may be used in conjunction with other metal chelators to add selectivity. Masking agents are used as an auxiliary complexing agent to prevent the complexation of certain metals so that others can be complexed. Examples of masking agents include sulfosalicylate which masks Al³⁺, cyanide which masks Co²⁺, Ni²⁺, Cu²⁺, Cd²⁺ and Zn^{2 +}, thiourea which masks Cu²⁺, citrate which masks Al³⁺, Sn⁴⁺ and Zr⁴⁺, and iodide which masks Hg2+.

The following table indicates some of the common metal complexers and some of the cations with which they form complexes:

| | | Color | Charge | No charge | |
|---|---|---|---|---|---|
| Complexer | Extraction | Forming | neutralization | neutralization | Representative ions complexed |
| 2-Aminoperimidine | x | | | x | SO₄²⁻, Ba²⁺ |
| hydrochloride | | | | | |
| 1-Phenyl-3-methyl-4- | x | | | x | Pu⁴⁺, UO₂²⁺ |
| benzoylpyrazolin-5-one | | | | | |
| Eriochrome black T | | x | x | | Ca²⁺, Mg²⁺, Sr, Zn, Pb |
| Calmagite | | x | x | | Ca²⁺, Mg²⁺, Sr, Zn, Pb |
| o,o-Dihydroxyazobenzene | | x | x | | Ca²⁺, Mg²⁺ |
| Pyridylazonaphthol (PAN) | | x | x | | Bi, Cd, Cu, Pd, Pl, Sn²⁺, UO₂²⁺, |
| | | | | | H_{g}²⁺, Th, Co, Pb, Fe²⁺, Fe³⁺, Ni²⁺, |
| | | | | | Zn²⁺, La⁺³ |
| Pyridylazonaphthol (PAN) | x | | x | | Alkali metals, Zr⁴⁺, Ge, Ru, Rh, Ir, |
| | | | | | Be, Os |
| Pyridylazo-resorcinol (PAR) | | x | x | | ReO₄⁻, Bi, Cd, Cu, Pd, Pl, Sn²⁺, |
| | | | | | UO₂²⁺, Hg²⁺, Th, Co, Pb, Fe²⁺, Fe^{3+,} |
| | | | | | Ni²⁺, Zn²⁺, La³⁺ |
| Thiazolylazo resorcinol (TAR) | | x | x | | Pb |
| 1,10-Phenanthroline | | x | | x | Fe²⁺, Zn, Co, Cu, Cd, SO₄²⁻ |
| 2,2′-Bipyridine | | x | | x | |
| Tripyridine | | x | | x | |
| Bathophenanthroline | | | | x | Cu²⁺, Cu⁺, Fe²⁺ |
| (4,7-diphenyl-1,10- | | | | | |
| phenanthroline) | | | | | |
| Bathophenanthroline | | x | | x | Cu²⁺, Cu⁺, Fe²⁺ |
| (4,7 diphenyl-2,9-dimethyl- | | | | | |
| 1,10-phenanthroline) | | | | | |
| Cuproine | | x | | x | Cu²⁺, Cu⁺, Fe²⁺ |
| Neocuproine | | x | | x | Cu²⁺, Cu⁺, Fe²⁺ |
| 2,4,6-Tripyridyl-S-triazine | | x | | | Fe²⁺ |
| Phenyl-2-pyridyl ketoxime | | x | | | Fe²⁺ |
| Ketoxime | | | x | | |
| Ferrozine | | x | x | | Fe²⁺ |
| Bicinchoninic acid | | | | x | Cu²⁺, Cu⁺ |
| 8-Hydroxyquinoline | x | | x | | Pb, Mg²⁺, Al³⁺, Cu, Zn, Cd |
| 2-Amino-6-sulfo-8- | | x | x | | |
| hydroxyquinoline | | | | | |
| 2-Methyl-8-hydroxyquinoline | x | | x | | Pb, Mg²⁺, Cu, Zn, Cd |
| 5,7-Dichloro 8-hydroxyquinoline | x | | x | | Pb, Mg²⁺, Al³⁺, Cu, Zn, Cd |
| Dibromo-8-hydroxyquinoline | x | | x | | Pb, Mg²⁺, Al³⁺, Cu, Zn, Cd |
| Naphthyl azoxine | | x | x | | |
| Xylenol orange | | x | x | | Th⁴⁺, Zr⁴⁺, Bi³⁺, Fe³⁺, Pb²⁺, Zn²⁺, |
| | | | | | Cu²⁺, rare earth metals |
| Calcein (Fluorescein-methylene- | | x | x | | Ca²⁺, Mg²⁺ |
| iminodiacetic acid) | | | | | |
| Pyrocatechol violet | | x | x | | Sn⁴⁺, Zr⁴⁺, Th⁴⁺, UO₂²⁺, Y³⁺, Cd²⁺ |
| Tiron (4,5-Dihydroxy-m- | | x | x | | Al³⁺ |
| benzenedisulfonic acid) | | | | | |
| Alizarin Red S | | x | x | | Ca²⁺ |
| (3,4-dihydroxy-2-anthra- | | | | | |
| quinonesulfonic acid) | | | | | |
| 4-Aminopyridine | x | | | x | |
| Thoron I | | x | | | |
| Arsenazo I | | x | x | | Ca²⁺, Mg²⁺, Th⁴⁺, UO₂²⁺, Pu⁴⁺ |
| Arsenazo III | | x | x | | Ca²⁺, Mg²⁺, Th⁴⁺, UO₂²⁺, Pu⁴⁺, Zr⁴⁺, |
| | | | | | Th⁴⁺ |
| EDTA (ethylenediamine | x | | x | | Fe²⁺, most divalent cations |
| tetraacetic acid) | | | | | |
| CDTA (cyclodiamine | x | | x | | Fe²⁺, most divalent cations |
| tetracetic acid) | | | | | |
| EGTA (ethylene glycol bis | x | | x | | Fe²⁺, most divalent cations |
| (β-aminoethylether)- | | | | | |
| N,N,N′,N′-tetraacetic acid) | | | | | |
| HEDTA (hydroxyethyl- | | | x | | Fe²⁺, most divalent cations |
| ethylenediamine triacetic acid) | | | | | |
| DPTA (diethylenetriamine | x | | x | | Fe²⁺, most divalent cations |
| pentaacetic acid) | | | | | |
| DMPS (dimercaptopropane | x | | x | | Fe²⁺, most divalent cations |
| sulfonic acid) | | | | | |
| DMSA | x | | x | | Fe²⁺, most divalent cations |
| (dimercaptosuccinic acid) | | | | | |
| ATPA (aminotrimethylene | x | | x | | Fe²⁺, most divalent cations |
| phosphonic acid) | | | | | |
| CHX-DTPA (Cyclohexyl | x | | x | | Fe²⁺, most divalent cations |
| diethylenetriaminopenta-acetate) | | | | | |
| Citric acid | x | | x | | Fe²⁺ |
| 1,2-bis-(2-amino-5- | | x | x | | Ca²⁺, K⁺ |
| fluorophenoxy)ethane- | | | | | |
| N,N,N′,N′-tetraacetic | | | | | |
| acid (5F-BAPTA) | | | | | |
| Desferoxamine | | | | | Fe²⁺ |
| Hydroquinone | x | | x | | Fe²⁺ |
| Benzoquinone | x | | x | | Fe²⁺ |
| dipicrylamine | x | | x | | K⁺ |
| Sodium tetraphenylboron | x | | x | | K⁺ |
| 1,2-dioximes | x | | x | | Ni²⁺, Pd²⁺, Mn²⁺, Fe²⁺, Co²⁺, Ni²⁺, |
| | | | | | Cu²⁺, Zn²⁺ |
| Alpha-furil dioxime | x | | x | | Ni²⁺, Pd²⁺, Mn²⁺, Fe²⁺, Co²⁺, Ni²⁺, |
| | | | | | Cu²⁺, Zn²⁺ |
| Cyclohexanone oxime | x | | x | | Ni²⁺, Pd²⁺, Mn²⁺, Fe²⁺, Co²⁺, Ni²⁺, |
| | | | | | Cu²⁺, Zn²⁺ |
| Cycloheptanone | x | | x | | Ni²⁺, Pd²⁺, Mn²⁺, Fe²⁺, Co²⁺, Ni²⁺, |
| | | | | | Cu²⁺, Zn²⁺ |
| Methyl cyclohexanone-dioxime | | x | x | | Ni²⁺, Pd²⁺, Mn²⁺, Fe²⁺, Co²⁺, Ni²⁺, |
| | | | | | Cu²⁺, Zn²⁺ |
| Ethyl cyclohexanone-dioxime | | x | x | | Ni²⁺, Pd²⁺, Mn²⁺, Fe²⁺, Co²⁺, Ni²⁺, |
| | | | | | Cu²⁺, Zn²⁺ |
| Isopropyl 4- | | x | x | | Ni²⁺, Pd²⁺, Mn²⁺, Fe²⁺, Co²⁺, Ni²⁺, |
| cyclohexanonedioxime | | | | | Cu²⁺, Zn²⁺ |
| Cupferron | x | | x | | M⁴⁺, M⁵⁺, M⁶⁺, Zr⁴⁺, Ga³⁺, Fe³⁺, Ti⁴⁺, |
| | | | | | Hf⁴⁺, U⁴⁺, Sn⁴⁺, Nb⁵⁺, Ta⁵⁺, V⁵⁺, |
| | | | | | Mo⁶⁺, W⁶⁺, Th⁴⁺, Cu²⁺, Bi³⁺ |
| N-Benzolyphenylhydroxyl-amine | | | x | | Sn⁴⁺, Zr⁴⁺, Ti⁴⁺, Hf⁴⁺, Nb⁵⁺, Ta⁵⁺, |
| (BPHA) | | | | | V⁵⁺, Mo⁶⁺, Sb⁵⁺ |
| Arsonic acids | x | | x | | Zr⁴⁺, Ti⁴⁺ |
| Mandelic acid | x | | x | | Zr⁴⁺, Hf⁴⁺ |
| Alpha-nitroso-beta-napthol | x | | x | | Co²⁺, Co³⁺ |
| Anthranilic acid | x | | x | | Ni²⁺, Pb²⁺, Co, Ni²⁺, Cu²⁺, Zn²⁺, Cd, |
| | | | | | Hg²⁺, Ag⁺ |
| Alpha-benzoinoxime | x | | x | | Cu²⁺ |
| Thionalide | x | | x | | Cu²⁺, Bi³⁺, Hg, As, Sn⁴⁺, Sb⁵⁺, Ag⁺ |
| Tannin | x | | x | | Nb, Ta |
| Ammonium oxalate | x | | x | | Th⁴⁺, Al³⁺, Cr, Fe²⁺, V⁵⁺, Zr⁴⁺, U⁴⁺ |
| Diethyldithio-carbamates | x | | x | | K⁺, most metals |
| 2-Furoic acid | x | | x | | Th⁴⁺ |
| Dimethylglyoxime (DMG) | x | | x | | Ni²⁺, Fe²⁺, Co²⁺, Al³⁺ |
| Isooctylthioglycolic acid | x | | x | | Al³⁺, Fe²⁺, Cu²⁺, Bi³⁺, Sn⁴⁺, Pb²⁺, |
| | | | | | Ag⁺, Hg²⁺ |

The listing of cations in this table should not be taken to be exclusive. Many of these agents will complex to some extent with many metal cations.

Among the chelating agents which may be useful for the practice of the current invention are monomeric polyacids such as EDTA, cyclohexanediamine tetraacetic acid (CDTA), hydroxy-ethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, ophthalmologically acceptable salts thereof, and combinations of any of the foregoing. Other exemplary chelating agents include: phosphates, e.g., pyrophosphates, tripolyphosphates, and hexametaphosphates; chelating antibiotics such as chloroquine and tetracycline; nitrogen-containing chelating agents containing two or more chelating nitrogen atoms within an imino group or in an aromatic ring (e.g., diimines, 2,2′-bipyridines, etc.); and polyamines such as cyclam (1,4,7,11 -tetraazacyclotetradecane), N-(C1-C30 alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), and deferoxamine (N′-[5-[[4-[[5-(acetylhydroxyamino)pentyl]amino]-1, 4-dioxobutyl]hydroxyamino]pentyl]-N′-(5-aminopentyl)-N-hydroxybutanediamide; also known as desferrioxamine B and DFO).

EDTA and ophthalmologically acceptable EDTA salts are particularly preferred, wherein representative ophthalmologically acceptable EDTA salts are typically selected from diammonium EDTA, disodium EDTA, dipotassium EDTA, triammonium EDTA, trisodium EDTA, tripotassium EDTA, and calcium disodium EDTA.

Without wishing to be bound by theory, it appears that a significant role played by the biocompatible metal complexer in the present formulations is in the removal of the active sites of metalloproteinases in the eye by sequestration of the enzymes' metal center. By inactivating metalloproteinases in this way, the metal complexer may slow or stop the degeneration of protein complexes within the eye, thereby providing an opportunity for the ocular tissues to rebuild themselves. In addition, by complexing with metal ions such as copper, iron, and calcium, which are critical to the pathways for formation and proliferation of free radicals in the eye, the metal complexer forms complexes that are flushed into the bloodstream and excreted renally. In this way, the production of oxygen free radicals, reactive oxygen species (ROS), and reactive molecular fragments is reduced, in turn reducing pathological lipid peroxidation of cell membranes, DNA, enzymes, and lipoproteins.

It is believed that under oxidative stress (such as occurs in diabetes), free radicals initiate peroxidation of membrane lipids, e.g. arachidonic acid (PUFA). This process forms highly reactive and toxic lipid aldehydes (LDAs). A major product is 4-hydroxynonenal (HNE), which is highly reactive and cytotoxic at micromolar concentrations. HNE is particularly deleterious to membrane proteins. It has been associated with opacification of lenses and apoptosis in human lens epithelial cells. Protein-HNE adducts form, which may result in membrane fluidity increase and Ca²⁺ influx. Caspases are activated which in turn leads to apoptosis.

Accordingly, the metal complexer is believed to be multifunctional in the context of the present invention, insofar as the agent serves to decrease unwanted proteinase (e.g., collagenase) activity, prevent formation of lipid deposits, and/or reduce lipid deposits that have already formed.

The formulation also includes an effective amount of a transport enhancer that facilitates penetration of the formulation components through cell membranes, tissues, and extra-cellular matrices, including the cornea. Suitable transport enhancers include, by way of example, substances having the formula 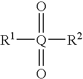
wherein R¹ and R² are independently selected from C₁-C₆ alkyl (preferably C₁-C₃ alkyl), C₁-C₆ heteroalkyl (preferably C₁-C₃ heteroalkyl), C₆-C₁₄ aralkyl (preferably C₆-C₈ aralkyl), and C₂-C₁₂ heteroaralkyl (preferably C₄-C₁₀ heteraralkyl), and Q is S or P. Within this class, those compounds wherein Q is S and R¹ and R² are C₁-C₃ alkyl are particularly preferred.

Suitable transport enhancers also include methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

MSM is an odorless, highly water-soluble (34% w/v at 79° F.) white crystalline compound with a melting point of 108-110° C. and a molecular weight of 94.1 g/mol. MSM is thought to serve as a multifunctional agent herein, insofar as the agent not only increases cell membrane permeability, but may also facilitate the transport of one or more formulation components to both the anterior and posterior of the eye. Furthermore, MSM per se is known to provide medicative effects, and can serve as an anti-inflammatory agent as well as an analgesic. MSM also acts to improve oxidative metabolism in biological tissues, and is a source of organic sulfur, which may assist in the reduction of scarring. MSM additionally possesses beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for formation of van der Waals associations, i.e., between the methyl groups and nonpolar (e.g., hydrocarbyl) segments of other molecules.

Again without wishing to be bound by theory, it is believed that the transport enhancer in formulations of the invention may assist in the process of transport of the metal complexer, not just across biological membranes, but also to the site at which it operates. It is possible that the transport enhancer and metal complexer may form a stable moiety which is more easily able to penetrate protein or lipid aggregates and remove metal ions which provide stability to those aggregates.

The transport enhancer of the formulations of the invention may contain more than one transport-enhancing substance. For example, a formulation of the invention can contain added DMSO. Since MSM is a metabolite of DMSO (i.e., DMSO is enzymatically converted to MSM), incorporating DMSO into an MSM-containing formulation of the invention will tend to gradually increase the fraction of MSM in the formulation. DMSO may also serve as a free radical scavenger, thereby reducing the potential for oxidative damage.

A factor which appears to be related to the performance of the formulations of the invention is the molar ratio of the transport enhancer to the metal complexer. A molar ratio of at least about 2, preferably at least about 4, more preferably at least about 8, is desirable. This may be on account of the formation of further complexes between transport enhancer and metal complexer which facilitate the latter's movement to the location of metal cations.

The concentration of the transport enhancer and metal complexer in the formulation are also of interest. In general concentrations on the order of a few percent by weight are preferred in aqueous vehicles, for example from about 1% to about 8%, more preferably from about 2% to about 6%. For example, where the transport enhancer is MSM and the metal complexer is EDTA, a concentration of about 2.5 wt % EDTA and about 5 wt % MSM is preferred.

The pharmaceutically acceptable carrier of the formulations of the invention may comprise a wide variety of non-active ingredients which are useful for formulation purposes and which do not materially affect the novel and useful properties of the invention. Reference is made to the relevant chapters of Remington's, cited above. In carriers that are at least partially aqueous one may employ thickeners, isotonic agents, buffering agents, and preservatives, providing that any such excipients do not interact in an adverse manner with any of the formulation's other components. It should also be noted that preservatives are not necessarily required in light of the fact that the metal complexer itself may serve as a preservative, as for example EDTA which has been widely used as a preservative in ophthalmic formulations.

Suitable thickeners will be known to those of ordinary skill in the art of ophthalmic formulation, and include, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers). The preferred amount of any thickener is such that a viscosity in the range of about 15 cps to 25 cps is provided, as a solution having a viscosity in the aforementioned range is generally considered optimal for both comfort and retention of the formulation in the eye. Any suitable isotonic agents and buffering agents commonly used in ophthalmic formulations may be used, providing that the osmotic pressure of the solution does not deviate from that of lachrymal fluid by more than 2-3% and that the pH of the formulation is maintained in the range of about 6.5 to about 8.0, preferably in the range of about 6.8 to about 7.8, and optimally at a pH of about 7.4. Preferred buffering agents include carbonates such as sodium and potassium bicarbonate.

The pharmaceutically acceptable ophthalmic carrier used with the formulations of the invention may be of a wide range of types known to those of skill in the art. For example, the formulations of the invention can be provided as an ophthalmic solution or suspension, in which case the carrier is at least partially aqueous. The formulations may also be ointments, in which case the pharmaceutically acceptable carrier comprises an ointment base. Preferred ointment bases herein have a melting or softening point close to body temperature, and any ointment bases commonly used in ophthalmic preparations may be advantageously employed. Common ointment bases include petrolatum and mixtures of petrolatum and mineral oil.

The formulations of the invention may also be prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® tradename from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5 μm to about 10 μm.

The formulations may also be incorporated into a sterile ocular insert that provides for controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art, and are typically composed of a water-swellable, gel-forming polymer such as collagen, polyvinyl alcohol, or a cellulosic polymer. Another type of insert that can be used to deliver the present formulation is a diffusional implant in which the formulation is contained in a central reservoir enclosed within a permeable polymer membrane that allows for gradual diffusion of the formulation out of the implant. Osmotic inserts may also be used, i.e., implants in which the formulation is released as a result of an increase in osmotic pressure within the implant following application to the eye and subsequent absorption of lachrymal fluid.

The invention also pertains to ocular inserts for the controlled release of combinations of the metal complexer and transport enhancer. These ocular inserts may be implanted into any region of the eye, including the sclera and the anterior and posterior segments. One such insert is composed of a controlled release implant containing a formulation that consists essentially of the biocompatible metal complexer, preferably EDTA or an ophthalmologically acceptable salt thereof, a transport enhancer, and a pharmaceutically acceptable carrier. The insert may be a gradually but completely soluble implant, such as may be made by incorporating swellable, hydrogel-forming polymers into an aqueous liquid formulation. The insert may also be insoluble, in which case the agent is released from an internal reservoir through an outer membrane via diffusion or osmosis.

It is to be understood that while the invention has been described in conjunction with the preferred specific embodiments thereof, the foregoing description and the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXAMPLE 1

### Prevention of Cataractogenesis in Diabetic Rats

Male Sprague-Dawley rats weighing 75-100 g were obtained from Central Animal Care Services at the University of Texas Medical Branch. The NIH guidelines and ARVO statement for the Use of Animals in Ophthalmic and Vision Research were strictly followed for the welfare of the animals.

Twenty-four rats were randomly assigned to six groups, each group having four rats. Intraperitoneal injections of streptozotocin (STZ) were used for diabetic induction in five of the six groups. STZ at a dosage of 70 mg/kg body weight was diluted in PBS buffer vehicle (pH 7.0). One group of control animals received an injection of PBS buffer alone. Animals were allowed to adjust to their diabetic state for 4 days.

Four days post-STZ administration, blood glucose levels were assessed in a glucose meter. A distal tail snip generated the 5 μl quantity of blood necessary for analysis. Weekly glucose levels were determined at 9 AM by removing the scab formed on the tail.

Eyedrop administration. Fresh eye drops were made weekly and kept at 40° C. Application of eye drops was initiated 15 days after the onset of diabetes, a time when diabetes-induced initial changes in the lens become evident. 8 μl of each eye drop was applied daily onto the cornea of the rat eye. The experiment was conducted for 70 days.

The treatments applied to the different groups of animals were as follows:
Non-Diabetic Control: 0.9% saline only.
Group 1 (Diabetic Control): 0.9% saline only.
Group 2: 0.1% MSM+0.1% EDTA
Group 3: 0.54% MSM+0.25% EDTA
Group 4: 0.54% MSM+0.5% EDTA
Group 5: 0.54% MSM
   The percentages indicated here are all by weight.

Lens collection and examination. Rats were sacrificed using 100% carbon dioxide at a low flow rate (25-30% of the volume of the cage per minute) with two rats in a cage. After the rats had stopped breathing for about 2 minutes, the rat eyeballs were removed and the lenses dissected. The epithelium with the capsule was removed under a surgical microscope and mounted on a glass slide (cells facing up). The slides were fixed in 4% paraformaldehyde for 15 minutes, transferred in 75% alcohol, and kept at 4° C. until use.

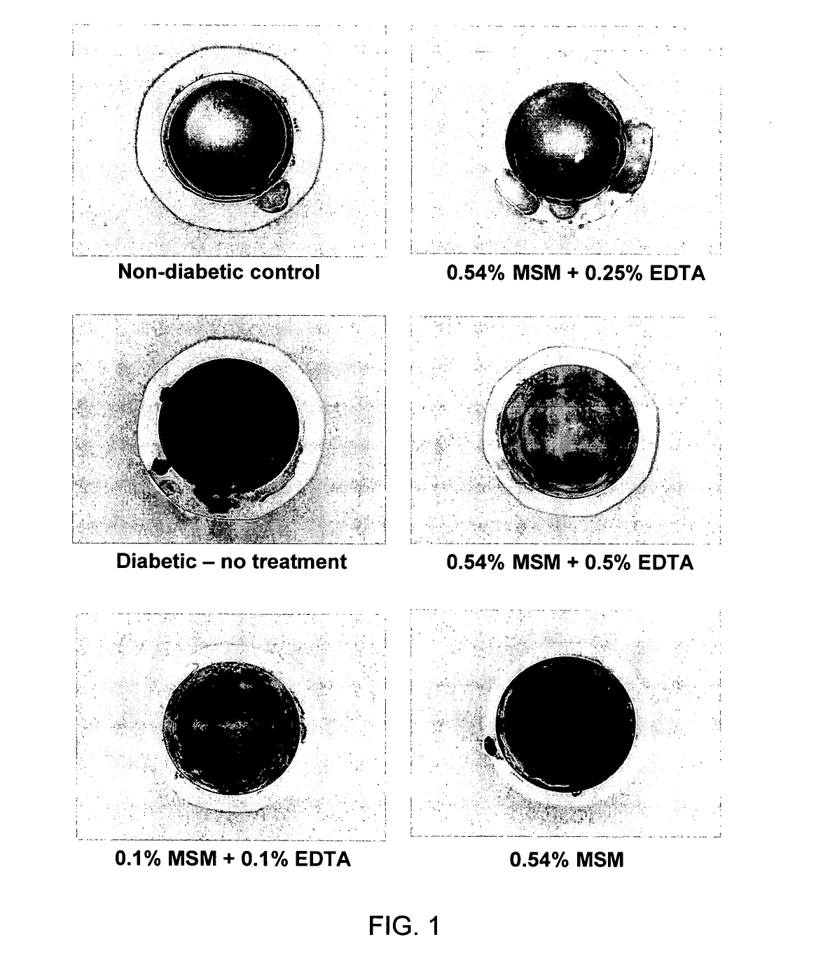

The lenses from each group of rats were examined and representative images were acquired using an inverted microscope (FIG. 1). The combination of 0.54% MSM and 0.25% EDTA appeared to be particularly effective in preventing cataractogenesis.

### EXAMPLE 2

### Evaluation Of Glucose-Induced Toxicity In Rat Lens Organ Culture (RLCE)

Animals. Male Sprague-Dawley rats weighing 200-250 g were obtained from Central Animal Care Services at the University of Texas Medical Branch. The NIH guidelines and ARVO statement for the Use of Animals in Ophthalmic and Vision Research were strictly followed for the welfare of the animals.

Rats were sacrificed with using 100% carbon dioxide at a low flow rate (25-30% of the volume of the cage per minute) with two rats in a cage. After the rats stopped breathing for about 2 minutes, the eyeballs were removed.

Preparation of Reagents.
Medium 199+0.1% Gentamicin: 250 ml of M199+250 μl of Gentamicin.
400 mM MSM (FW 94.2): 376 mg MSM+PBS to final volume to 10 ml.
50 mM EDTA (Tetrasodium Salt FW 380): 190 mg EDTA+PBS 8 ml, adjust pH to 7.2 with HCI.
Adjust final volume to 10 ml.
2.5 M Glucose (FW 180): 900 mg glucose+2 ml dd H₂O

Experimental Procedure. 1. Sacrificed four rats, removed the eyeballs as soon as possible and put them into a tube containing PBS with 0.1% gentamicin. 2. Dissected the lenses immediately and washed with 1% penicillin/streptomycin in sterile with PBS. 3. Transferred each lens to a well of a 12-well plate (2 ml of medium per well, i.e., per lens). Each treatment was performed in 2 wells. The lenses were cultured in medium 199 containing 0.1% gentamicin at 37° C. in a 5% CO₂ humidified atmosphere.

Reagents as described above were added to three groups of two wells to give the following three treatments:
50 mM glucose
50 mM glucose+4 mM MSM
50 mM glucose+4 mM MSM+0.5 mM EDTA

One group of two wells was left untreated as a control. The medium and the reagents were changed every day. After seven days, the lenses were visualized under a Nikon Eclipse 200. Photographs were taken using a Multidimensional Imaging System, and the level of light transparency through the lenses was determined.

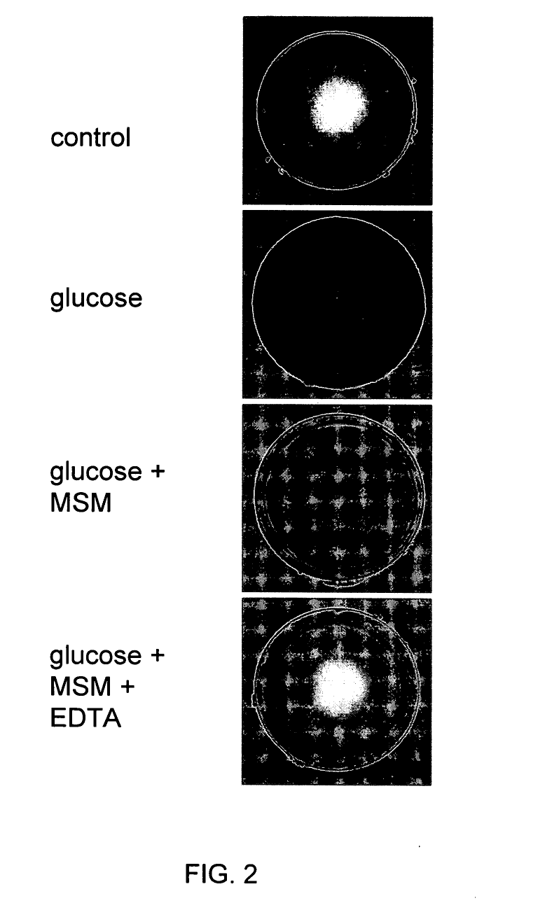

Results. Photographs of the lens culture showed that significant rat lens opacity was induced with glucose (FIG. 2). MSM mitigated lens opacification by glucose; MSM plus EDTA provided the most effective protection.

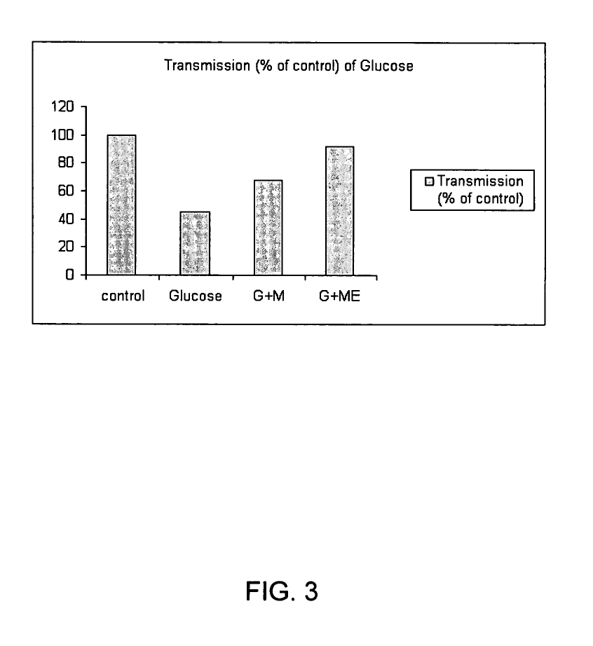

The level of light transmission through the lens was used to quantify lens opacity for each treatment. Consistent with the photographic results, MSM improved the level of light transmission, while MSM+EDTA gave an even greater improvement (FIG. 3). Light transmission through the lens treated with glucose was only 45% of light transmission through the untreated control. Light transmission through the lenses treated with glucose plus MSM (G+M) and glucose and MSM/EDTA (G+ME) were 68% and 92% respectively.

### EXAMPLE 3

### Effect of MSM and MSM/EDTA on Viability of Human Lens Epithelial Cells (HLEC) Subjected to Glucose-Induced Toxicity

Materials. EDTA (Tetrasodium Salt), ferrous ammonium sulfate, ferric chloride, adenosine 5′-diphosphate (ADP), ascorbic acid, and H₂O₂ were purchased from Sigma. All cell culture medium components were from Invitrogen.

Cell Culture and Treatment. Human lens epithelial cells (HLECs) with extended life span were cultured in DMEM medium containing 0.1% gentamicin and supplemented with 20% fetal bovine serum at 37° C. in a 5% CO₂-humidified atmosphere. 1.0×10⁵HLECs/ml (Passage 5) were seeded in 12-well plate overnight prior to the addition of glucose, MSM or MSM/EDTA. The wells were divided into six groups of two wells.

Cell viability. Cell survival was determined by Trypan Blue staining and counting with a hemocytometer. Dead cells stain blue, while live cells exclude Trypan Blue. Cell viability is represented as a percentage corresponding to the number of live cells divided by the total number of cells.

Preparation of Reagents.
HLEC medium: DMEM+20% FBS +0.1% gentamicin
400mM MSM: 376 mg/10 ml PBS for stock
50mM EDTA (Tetrasodium Salt): 190 mg/10 ml PBS for stock, pH 7.2
5 M Glucose: 1800 mg/10 ml of dd H₂O

Experimental Procedure. 1. Seeded 0.5×10⁵ /ml of HLEC (Passage 5) into three 12-well plates, then incubated at 37° C. overnight. 2. Changed medium to 2% FBS DMEM medium. 3. Added glucose, MSM, or MSM/EDTA to the proper wells, so as to achieve final concentrations as follows:
50 mM glucose (well groups 1, 2, 3)
4 mM MSM (well groups 2, 3, 5, and 6)
0.5 mM EDTA (well groups 3 and 6)

After adding glucose, MSM, and EDTA, the cells were incubated at 37° C. with 5% CO₂ and 95% air for 16 hrs. They were harvested with 0.25% Trypsin-EDTA and cell viability was determined with Trypan-Blue.

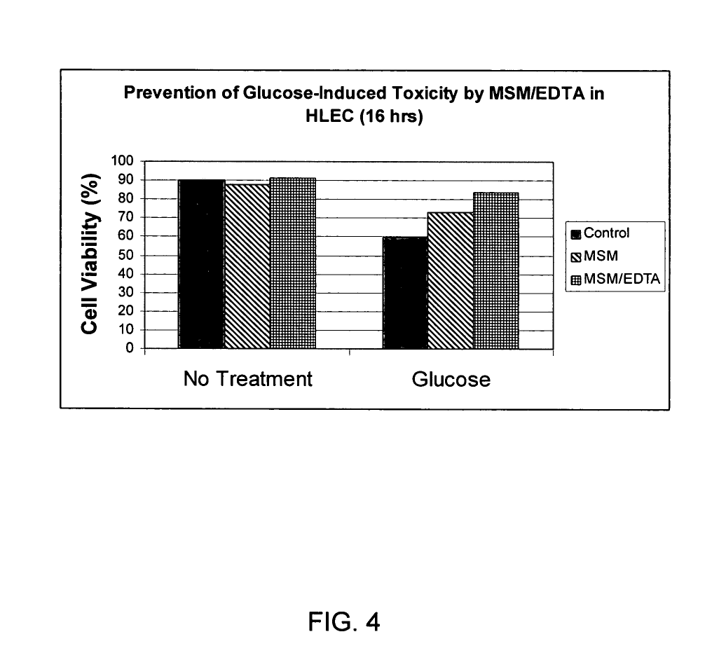

Results. FIG. 4 shows the percent of cell viability under each condition. Glucose decreased cell viability by 30%. The addition of 4 mM MSM increased the percent cell viability, while the addition of 4 mM MSM with 0.5 mM EDTA gave a greater increase in the percentage of viable cells. A Chi Square test demonstrated the protective effect of MSM/EDTA was statistically significant (P value of less than 0.05).

We claim:

1. A method of treating the ophthalmic complications of diabetes, comprising the step of administering to a patient with diabetes, insulin resistance, or a risk factor for diabetes an effective amount of a pharmaceutical formulation comprising a transport enhancer and a biocompatible metal complexer in a pharmaceutically acceptable carrier.

2. The method of claim 1, wherein the carrier is at least partly aqueous.

3. The method of claim 1, wherein the transport enhancer has molecular weight less than 200 daltons.

4. The method of claim 1, wherein the transport enhancer also serves to improve oxidative metabolism in the body.

5. The method of claim 1, wherein the transport enhancer can scavenge free radicals.

6. The method of claim 1, wherein the transport enhancer comprises a compound of the formula 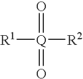
wherein R₁ and R² are independently selected from C₁-C₆ alkyl, C₁-C₆ heteroalkyl, C₆-C₁₄ aralkyl, and C₂-C₁₂ heteroaralkyl, and Q is S or P.

7. The method of claim 6, wherein Q is S.

8. The method of claim 6, wherein R¹ and R² are C₁-C₃ alkyl.

9. The method of claim 1, wherein the transport enhancer comprises DMSO or MSM or a combination of both.

10. The method of claim 1, wherein the transport enhancer comprises MSM.

11. The method of claim 1, wherein the metal complexer is a chelating agent.

12. The method of claim 11, wherein the metal complexer is selected from ethylenediamine tetraacetic acid (EDTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, ophthalmologically acceptable salts thereof, and combinations of any of the foregoing.

13. The method of claim 12, wherein the metal complexer is selected from EDTA and ophthalmologically acceptable salts thereof.

14. The method of claim 1, wherein the molar ratio of the transport enhancer to the metal complexer is at least about 2.

15. The method of claim 8, wherein the molar ratio of the transport enhancer to the metal complexer is at least about 4.

16. The method of claim 8, wherein the molar ratio of the transport enhancer to the metal complexer is at least about 8.

17. The method of claim 1, wherein the step of administering is performed by means of eye drops.

18. The method of claim 1, wherein the step of administering is performed by means of an ocular insert.

19. The method of claim 1, wherein the transport enhancer comprises at least about 0.5% by weight of the pharmaceutical formulation which is administered.

20. The method of claim 1, wherein the metal complexer comprises at least about 0.25% by weight of the pharmaceutical formulation which is administered.

21. A sterile ophthalmic formulation consisting essentially of
(a) a biocompatible metal complexer,
(b) a transport enhancer,
(c) an optional additional transport enhancer,
(d) a pharmaceutically acceptable carrier, and
(e) other optional excipients.

22. The formulation of claim 21, wherein the transport enhancer is a compound of the structure 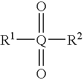
wherein R¹ and R² are independently selected from C₁-C₆ alkyl, C₁-C₆ heteroalkyl, C₆-C₁₄ aralkyl, and C₂-C₁₂ heteroaralkyl, and Q is S or P.

23. The formulation of claim 22, wherein Q is S.

24. The formulation of claim 23, wherein R¹ and R² are C₁-C₃ alkyl.

25. The formulation of claim 21, wherein the metal complexer is selected from ethylenediamine tetraacetic acid (EDTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, ophthalmologically acceptable salts thereof, and combinations of any of the foregoing.

26. The formulation of claim 25, wherein the metal complexer is selected from EDTA and ophthalmologically acceptable salts thereof.
